(19) United States
(12) Patent Application Publication
(10) Pub. No.: US 2009/0012014 A1
(43) Pub. Date: Jan. 8, 2009

(54) Compound, Composition and a Process Thereof

(75) Inventors: Sunil Bhaskaran, Wahorie (IN); Mohan Vishwaraman, Kothrud (IN)

Correspondence Address: BUCHANAN, INGERSOLL & ROONEY PC, POST OFFICE BOX 1404, ALEXANDRIA, VA, 22313-1404, US

(73) Assignee: INDUS BIOTECH PVT. LTD, Maharashtra (IN)

(21) Appl. No.: 11/833,520

(22) Filed: Aug. 3, 2007

(30) Foreign Application Priority Data
Jul. 2, 2007 (IN) 01251/MUM/2007

(51) Int. Cl. A61K 31/704; A61P 29/00; C07J 53/00; C07J 75/00

(52) U.S. Cl. 514/26; 536/5

(57) ABSTRACT

The present invention provides a novel steroidal glycoside for management of rheumatic and inflammatory disease conditions. In addition, the present invention also provides a process for preparation of steroidal glycoside from plant fenugreek. Further, the invention also provides a pharmaceutical composition of the steroidal glycoside for management of rheumatic disease conditions and its associated disorders.

### FIELD OF THE PRESENT INVENTION

The present invention is in relation to a method of preparing a composition from Trigonella Foenum Graecum. More particularly the present invention further elucidates the use of this composition for management of rheumatic diseases such as bursitis, tendonitis, rheumatoid arthritis, osteoarthritis, soft tissue rheumatism, spondylitis and back pain. This invention also refers to the management of inflammatory conditions.

### BACKGROUND OF THE INVENTION

Arthritis refers to inflammation of a joint from any cause. The diagnosis of arthritis suggests a progressive and possibly a crippling disease to many patients. However, to the physician arthritis simply means inflammation of the joints. There are approximately two hundred different types of rheumatic diseases (Disease of the joints and associated tissues such as muscles and bones). Rheumatic diseases can be classified broadly into the following four categories.
i) Inflammatory (Rheumatoid) Arthritis
ii) Soft Tissue Rheumatism
iii) Osteoarthritis
iv) Spondylitis and Back Pain

At the ends of bones in a human or mammalian body, there is a smooth layer of cartilage which acts as a shock absorber, when we move and jump about. Surrounding the joint between two bones, there is a membrane called synovial membrane which produces a viscous fluid “Synovial fluid” to nourish and lubricate the cartilage. There is a thick layer of ligaments around synovial membrane which helps to keep the bones stable and in place relative to each other. There are tendons outside this ligament layer which attach the muscles to the bones.

Rheumatoid arthritis (RA) is an autoimmune disease which means the immune system of the body treats the joints and connective tissues of joints as foreign and triggers an autoimmune attack directed against the joints. The reason why this happens is unknown. The immune cells start secreting cytokines which cause gradual destruction and damage of cartilage and the bone underneath. The usual symptoms of this disease are pain, stiffness of joints, swelling of joints, movement disability, muscle weakness, mild fever and general feeling of being unwell. Blood tests normally show raised levels of inflammation (C-reactive protein) and an antibody called Rheumatoid Factor (RA factor).

The drug treatments used for rheumatoid arthritis are:
i) Pain Killers
ii) Non Steroidal Anti-inflammatory Drugs (NSAIDS)
iii) Selective COX-2 Inhibitors
iv) Disease Modifying Anti Rheumatic Drugs (DMARD)

Pain killers and NSAIDS are often used as the first line of treatment to provide symptomatic relief. Selective Cox2 inhibitors provide symptomatic relief to pain and inflammation without the accompanying side effects of pain killers and NSAIDS such as gastrointestinal toxicity.

DMARDS are more powerful drugs which can suppress the symptoms of arthritis and some of them suppressing the disease process itself thereby reducing the amount of joint damage. The drugs under this category are Leflunomide, Methotrexate, Azathioprine, Cyclophosphamide, Cyclosporin, Gold Compounds, d-Pencillamine, Antinalarial drugs etc.
v) Corticosteroids

These are used in severe advanced stage. They are more effective in suppressing immunity and related symptoms of arthritis. However, this class of drugs has very severe side effects.
vi) Biologics

These are products of recombinant technology of molecular biology. These drugs are designed to affect the biochemical pathways that cause inflammation of joint and joint damage. Interleukin-1 (IL-1) and Tumor Necrosis Factor (TNF-α) are two cytokines implicated in joint damage and destruction. The biological are designed to be the receptor antagonists for the cytokines.

However, one major disadvantage with all of these drugs is the side effects and toxicity profile. The pain killers and NSAIDS cause ulcers and are not tolerated by patients. Some of the COX-2 inhibitors have cardiac toxicity. Mostly the DMARD drugs are highly toxic and can not be used for a long time. Cortico steroids have very severe side effects such as high blood pressure, diabetes, osteoporosis, obesity, cataracts, stomach ulcers etc. Biological also have side effects wherein they cause opportunistic infection. There is a group of rheumatic conditions which cause inflammation of spine and joints such as ankylosing spondylitis. The most commonly used drug for ankylosing spondylitis is non-steroidal anti-inflammatory drug. Other methods of treatment are use of azathioprine and sulfasalazine. Radiotherapy is also used at times.

Osteoarthritis is the commonest of all arthritis. This occurs in about 20% of the population as a whole and in 50% of the people over sixty years of age. In this disease the surface of the joint is damaged and this is an abnormal reaction in the underlying bone. It is probably caused by the biochemical abnormalities of the cartilage and made worse by inflammatory conditions. The most popular treatment is nonsteroidal anti-inflammatory drugs. When the ostioarthritis is accompanied by a degree of joint inflammation, injection of steroids and hylans into the joints is used as a therapy Soft tissue rheumatism refers to several related conditions in which musculoskeletal pain arises not from joint or bones but from the soft tissues around them such as tendons. They include bursitis and tendonitis. These conditions can also include “Tennis Elbow and Housemaid's Knee”. Normal line of treatment is NSAIDS. Back pain is extremely common and varies greatly in severity. Drug treatments for back pain vary according to the cause of the pain. NSAIDS are commonly used for inflammatory conditions and back pain.

Thus, it is very clear that the usual drug available for rheumatic disease is NSAID. These drugs have severed side effects and inhibit long term use. Therefore, there is a need for “Kinder and Gentler” drug derived from plant matter for management of these conditions over a long term without the toxicity of long term use.

### PRIOR ART OF THE INVENTION

The related art of interest describes various processes for obtaining different therapeutic composition, but none discloses the present invention. The related art will be discussed in the order of perceived relevance to the present invention.

U.S. Pat. No. 5,707,631 of Jan. 13, 1998.

Therapeutic herbal composition by Chaim J. Liberman et al claims a composition of raw herbs containing Trigonella foemun-graecum for use in lowering cholesterol, treating arthritis, blood pressure and alzheimer's disease.

This patent describes the formulation of herbal composition consisting of Trigonella foemun-graecum. Syzvgium aromatium fruit, Allium sativum bulb. Cinnamon zeylanicum bark, Saussurea costus root and Euphorbia lathyrus bud. The inventors have shown evidence of this composition in reducing cholesterol, triglycerides and increase in HDL. However, there is no evidence which can be understood and practiced by anyone skilled in the art regarding any action of this composition in arthritis in this patent. Besides, this composition is a mixture of six different herbs. It is very difficult to establish that the Trigonella foenum-graecum is contributing to this effect at all.

U.S. Pat. No. 6,080,401 of Jun. 27, 2000.

Herbal and pharmaceutical drugs enhanced with probiolics by Malireddy S. Reddy et al describes a composition consisting mixtures of several herbs and mixtures of several probiotic preparations. One of the herbs referred to is Trigonella foenum-graecum. It is not possible to establish that the beneficial effect is caused by Trigonella foenum-graecum in the case of arthritis. Besides, this patent does not disclose the methodology used for treatment of arthritis and show the efficacy has been established in the case of arthritis.

U.S. Pat. No. 6,884,442 B2 Apr. 26, 2005.

Anti-inflammatory agents and food and drinks containing the same by Michio Tani et al describes a herbal composition consisting of either dried substances or extracts of Perilla leaf, Cocao, Fennel, Fenugreek seed, Rosemary, Junipers, Berry, and Celery seed. This combination has been shown to have anti-inflammatory effect. They have shown evidence of a clinical trial. However, the end point markers are subjective evaluation. Besides, as this is a combination of seven herbs and fenugreek seed is only one of them, it is very difficult to establish that fenugreek seed is solely contributing to this effect. The degree of efficacy has not been established to warrant to use in severe inflammatory conditions like rheumatic diseases.

US Patent Application No. US 2006/0105055 A1 of May 18, 2006.

Arthritis and muscle soothing formulation containing whole egg powder by Michael Marenick et al refers to the use of essential oil derived from fenugreek (Trigonella foenum-graecum) for their egg powder formulation. This invention does not disclose specifically the role of fenugreek oil for arthritis.

US Patent Application No. US 2006/0269621 A1 of Nov. 30, 2006.

Novel therapeutic extracts and molecules for degenerative conditions by Villo Morawala Patel describe an invention of plant extracts which have very wide activity including arthritis and pain. The inventor has included Trigonella foenum-graecum as one of the extracts. However, this application does not contain any specification that contains a written description of the invention that elucidates the application of this extract in arthritis and pain. It appears that the description is more related to insulin sensitization, insulin mimetic activity etc.

None of the above citations, taken either singly or in combination, is seen to describe the instant invention as claimed. Thus, obtaining compound of instant invention from Trigonella foenum graecum using the process of instant invention will therefore helps in addressing the problems associated with the prior art. The novelty resides not only in the compound but also in the process of instant invention. It is the sequence of steps involved which are unique and which has resulted in arriving at high yield and purity of the compound of instant invention.

### OBJECTS OF THE PRESENT INVENTION

The principal object of the present invention is to develop a purified composition with a well defined chemical marker for treating rheumatic and inflammatory diseases.

Another object of the present invention is to develop a process for the preparation of a purified composition from fenugreek seed (Trigonella foenum-graecum) for treating rheumatic diseases and inflammatory conditions.

Yet another object of the present invention is to isolate purify, identify and elucidate the structure of a chemical marker in this extract.

Still another object of the present invention is to develop a purified composition which is rich in the glycoside identified as 26-O-β-D Glucopyranosyl pseudodiosgenin-3-0-α-L-Rhamnopyranosyl 1-(1-2)-β-D Glucopyranoside having molecular weight of 914 and a chemical formula of C₄₇H₇₅O₁₇. This is contained in the final composition up to 70%.

Still another object of the present invention is to make use of this glycoside rich composition for treatment of rheumatic and inflammatory disease conditions.

### SUMMARY OF INVENTION

A compound of structural formula I

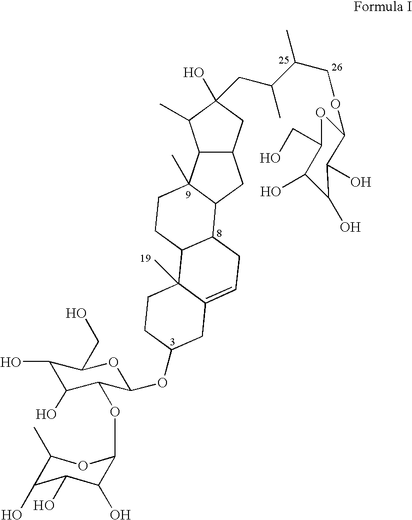

A composition comprising compound of structural formula I

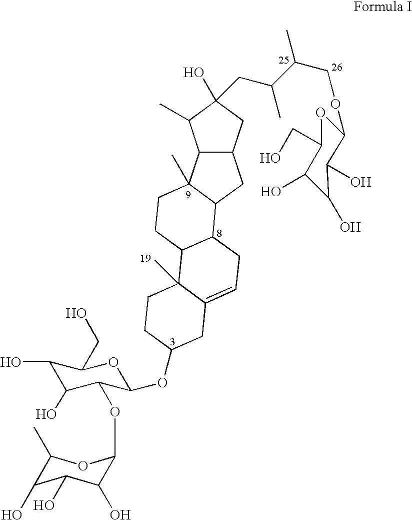

and pharmaceutically acceptable additive(s) for management of rheumatic and inflammatory disease conditions; a process for preparation of compound of structural formula I,

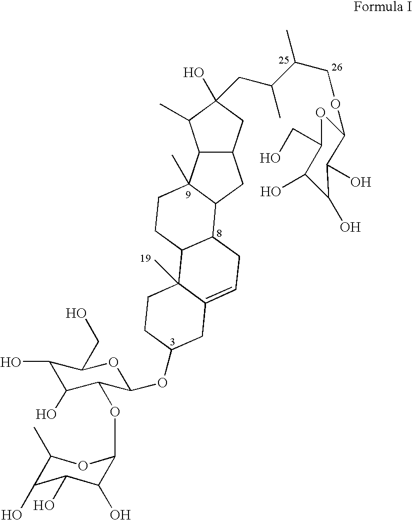

from plant Trigonella foenum-graecum, wherein said process comprises steps of
a. flaking fenugreek seeds;
b. extracting flaked seeds with n-hexane and hydro-alcohol to remove fatty matter;
c. concentrating the extract under vacuum to obtain a mass;
d. dissolving concentrated mass to obtain a clear solution;
e. passing clear solution through ion-exchange resin and adsorbent column to retain active compounds; and
f. eluting adsorbed active compounds rising hydro-alcohol and drying the eluant to obtain powder of the compound of formula I; and a method of treating rheumatic and inflammatory disease conditions in a subject in need thereof, said method comprising step of administering pharmaceutically effective amount of compound of structural formula I

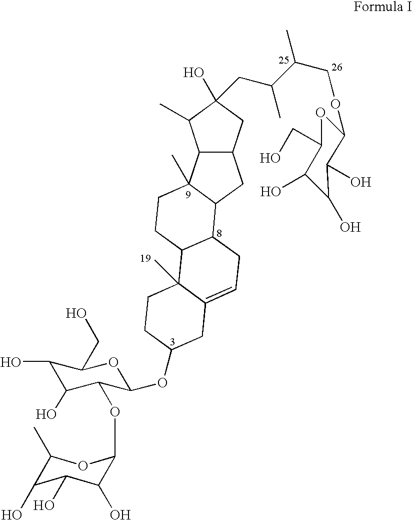

optionally along with pharmaceutically acceptable additives to the subject.

### BRIEF DESCRIPTION OF THE ACCOMPANYING DRAWINGS

FIG. 1 HPLC chromatogram of purified extract showing two marker compounds

FIG. 2 CRP (Nephlometry) response over time in patients completing 12 months study

FIG. 3 RF titer (Nephlometry) response over time in patients completing 12 months study

FIG. 4 HDL/LDL ratio over time in patients completing 12 months of follow up

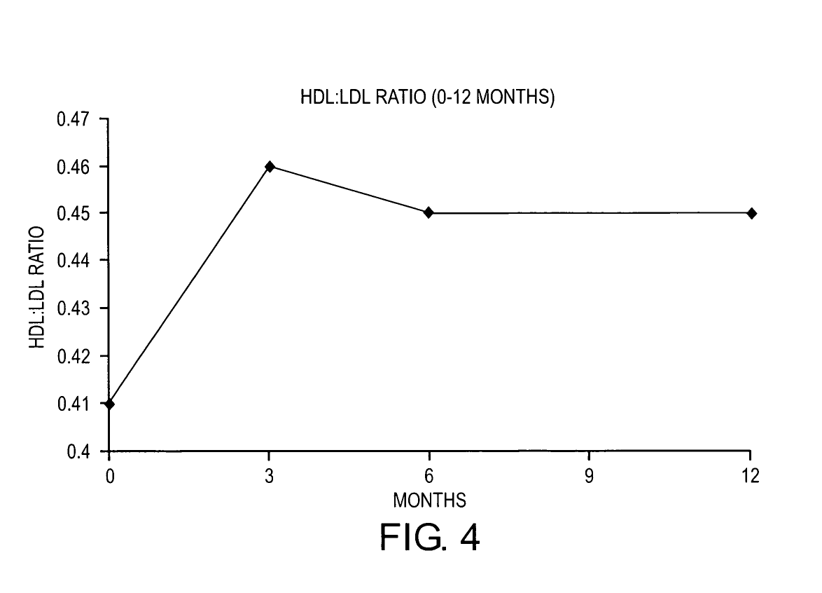

### DETAILED DESCRIPTION OF THE INVENTION

The present invention is in relation to a compound of structural formula I

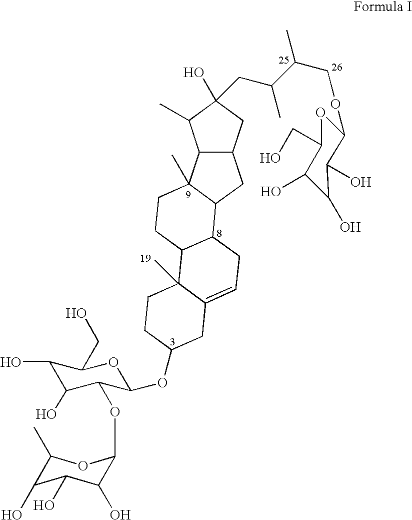

In another embodiment of the present invention, wherein said compound is chemically 26-0-β-D Glucopyranosyl pseudodiosgenin-3-0-α, -L-Rhamnopyranosyl 1-(1-2) -β-D Glucopyranoside.

In yet another embodiment of the present invention, wherein said compound is a steroidal glycoside having molecular weight of 914 and molecular formula of C₄₇H₇₈O₁₇.

In still another embodiment of the present invention, wherein said compound is obtained from plant Trigonella foenum graecum.

The present invention is in relation to a composition comprising compound of structural formula I

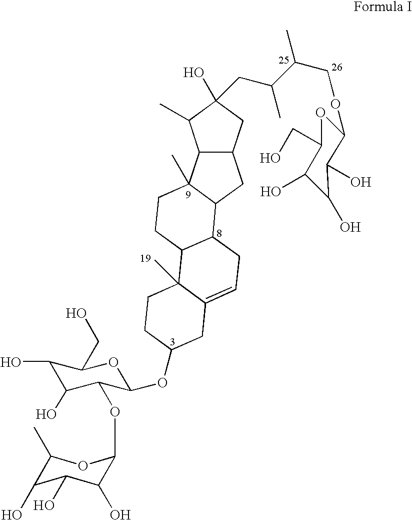

and pharmaceutically acceptable additive(s) for management of rheumatic and inflammatory disease conditions.

In another embodiment of the present invention, wherein the additives are selected from a group comprising granulating agents, binding agents, lubricating agents, disintegrating agents, sweetening agents, coloring agents, flavoring agents, coating agents, plasticizers, preservatives, suspending agents, emulsifying agents and spheronization agents.

In yet another embodiment of the present invention, wherein said composition is formulated into various dosage forms selected from a group comprising tablet, troches, lozenges, aqueous or oily suspensions, dispersible powders or granules, emulsion in hard or soft gel capsules, syrups, elixirs, phyotceuticals, neutraceuticals and food stuffs.

In still another embodiment of the present invention, wherein said compound is obtained from plant Trigonella foemun graecum.

In still another embodiment of the present invention, wherein said plant parts are roots, shoots, leaves, seeds, entire plant and preferably seeds.

In still another embodiment of the present invention, wherein said composition is non-toxic and free of side effects.

The present invention is in relation to a process for preparation of compound of structural formula I,

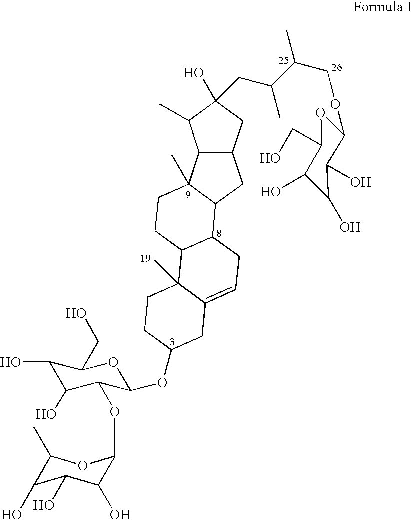

from plant Trigonella foenum-graecum, wherein said process comprises steps of
g. flaking fenugreek seeds;
h. extracting flaked seeds with n-hexane and hydro-alcohol to remove fatty matter;
i. concentrating the extract under vacuum to obtain a mass;
j. dissolving concentrated mass to obtain a clear solution;
k. passing clear solution through ion-exchange resin and adsorbent column to retain active compounds; and
l. eluting adsorbed active compounds using hydro-alcohol and drying the eluant to obtain powder of the compound of formula I.

In another embodiment of the present invention, wherein said extraction is carried out for a time period ranging from 8 to 12 hrs and preferably 10 hrs.

In yet another embodiment of the present invention, wherein said extraction is carried out at a temperature ranging from 30 to 40° C. and preferably 35° C.

In still another embodiment of the present invention, wherein the hydro alcohol for extraction is isopropyl alcohol and water in a ratio ranging from 50:50 to 80:20 and preferably 70:30,

In still another embodiment of the present invention, wherein said extract is concentrated under vacuum at a temperature ranging from 45 to 55° C. and preferably 50° C.

In still another embodiment of the present invention, wherein said mass is dissolved in deionized water.

In still another embodiment of the present invention, wherein said ion-exchange resin is cation exchange gel type resin.

In still another embodiment of the present invention, wherein said hydro alcohol for eluting adsorbed compounds is water and ethyl alcohol at an initial ratio of 30:70 followed by a shift to ratio of 10:90.

In still another embodiment of the present invention, wherein the yield of the compound of formula I

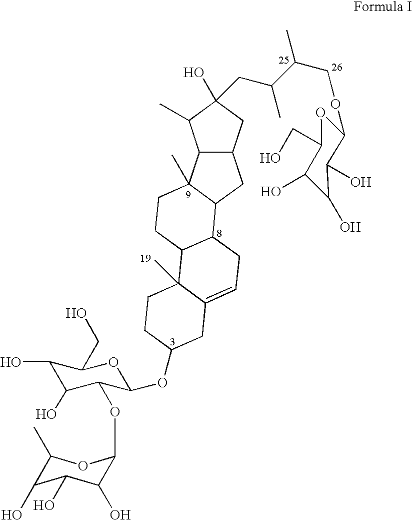

is ranging between 12 to 20 gms/500 gm of fenugreek seeds.

In still another embodiment of the present invention, wherein the purity of the compound of formula I is ranging from 50% to 70%.

The present invention is in relation to a method of treating rheumatic and inflammatory disease conditions in a subject in need thereof, said method comprising step of administering pharmaceutically effective amount of compound of structural formula I

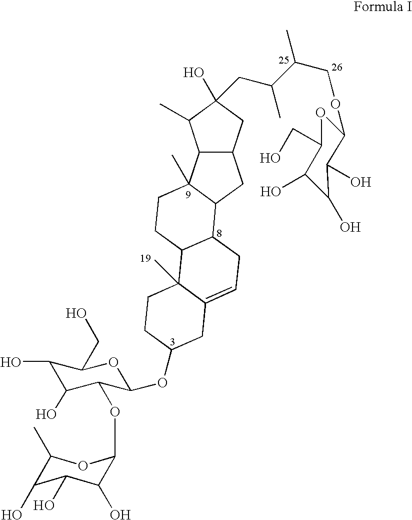

optionally along with pharmaceutically acceptable additives to the subject.

In another embodiment of the present invention, wherein said compound is effective in reducing disease related factors comprising Rheumatoid factor, Tumor Necrosis Factor -α, Blood Urea Nitrogen, and C reactive protein and increasing HDL/LDL ratio and albumin.

In yet another embodiment of the present invention, wherein said additives are selected from a group comprising granulating agents, binding agents, lubricating agents, disintegrating agents, sweetening agents, coloring agents, flavoring agents, coating agents, plasticizers, preservatives, suspending agents, emulsifying agents and spheronization agents.

In still another embodiment of the present invention, wherein said composition is formulated into various dosage forms selected from a group comprising tablet, troches, lozenges, aqueous or oily suspensions, dispersible powders or granules, emulsion in hard or soft gel capsules, syrups, elixirs, phyotceuticals, neutraceuticals and food stuffs.

In still another embodiment of the present invention, wherein the subject is an animal or human being.

In still another embodiment of the present invention, wherein the composition is administered at dosage ranging between 5 mg/kg to 20 mg/kg body weight and preferably 16 mg/kg body weight.

In still another embodiment of the present invention, wherein the composition is non-toxic and free of adverse effects including gastric ulcerogenicity.

In still another embodiment of the present invention, wherein said compound is obtained from plant Trigonella foenum graecum

In still another embodiment of the present invention, wherein said compound is obtained from plant parts are roots, shoots, leaves, seeds, entire plant and preferably seeds.

In still another embodiment of the present invention, wherein said rheumatic disease conditions comprise bursitis, tendonitis, rheumatoid arthritis, osteoarthritis, soft tissue rheumatism, spondylitis and back pain.

In still other embodiments the following are performed
Fenugreek seeds are flaked to expose the inner core so as to ensure effective defatting, extraction and processing
Hexane solvent is passed through the fenugreek bed repeatedly to achieve effective defatting of the fenugreek.
The flaked seeds are loaded in a percolater and solvent mixture comprising of an aliphatic alcohol comprising of one carbon atom to three carbon atoms and water are passed through tire fenugreek layer to achieve effective extraction of the, amino acids, saponin and other bioactive compounds.
The solvent is vacuum concentrated at lower temperatures to ensure the integrity of the mass and the resultant mass is dissolved in deionized water to get clear solution.
The clear solution thus obtained is passed through a strong acid cation exchange resin in Gel form to retain the amino acids and other acids.
The column outlet liquid is passed through an adsorbent column comprising of any one of the following commercial resins. SP-207, HP20SS, XAD 2 (halogenated).
The adsorbed material is eluted gradiently using aqueous alcohol to isolate the active compounds.
The above process is monitored by Thin layer chromatographic system comprising of toluene, ethylacetate, methanol, water in the ratio of 6:3:6:1.
The above isolated and purified material is spray dried into a flee flowing powder.
The above purified material is subjected to HPLC measurement as per procedure defined hereunder to obtain 2 marker compounds. The main marker is to the extent of 70%.
The above extracted is further fractionated and purified to get a pure fraction of marker compound 1.
This marker compound is subjected proton NMR and C-13 NMR to elucidate the structure of this glycoside.
The glycoside identified is 26-0-β-D Glucopyranosyl pseudodiosgenin -3-0-∝-L-Rhamnopyranosyl 1-(1-2)-β-D Glucopyranoside having a molecular weight of 914 and a chemical formula of C₄₇H₇₈O₁₇
This compound is tested for acute activity in Swiss Wistar rats to establish anti-inflammatory activity of 32.44% inhibition of paw edema in Carageneen induced models at 100 mg per kg dose.
This compound is tested in sub acute model of inflammation of cotton pallet granuloma in Swiss Wistar rats. This has an inhibition of granuloma of 32.3% at 100 mg per kg dosing.
This compound is tested in chronic model of arthritis of FCA (Freunds Complete Adjuvant) in Swiss Wistar rats. This has shown inhibition of 56% at 100 mg per kg dose.
The compound is tested in chronic model of arthritis of FCA and has shown decrease in C-reactive protein indicative of reduction in inflammation.
The compound is tested for ulcer formation in Swiss Wistar rats and found that it does not produce any stomach ulcer.
This compound is tested in one year human clinical study in patients with rheumatoid arthritis. It has shown significant disease modifying anti-arthritic action accompanied by reduction in all ACR core set criteria, C-reactive protein and RA factor.
This compound has led to remission of disease in 10 patients treated during the clinical trial.
This compound has demonstrated a significant increase in HDL/LDL ratio of rheumatic patients from 0.41 to 0.45. Considering the fact that rheumatic patients develop cardiovascular problem, this drug is very beneficial in alleviating this.
The test compound has shown, significant improvements in blood urea nitrogen and albumin confirming that the catabolic action of the rheumatoid arthritis is decreased.

Fenugreek seeds were flaked using roller flaking machine to Size of thickness varying between 1 mm to 4 mm size. The effective exposure of the inner core was achieved by flaking to a size preferably of 2 mm thickness. The flaked seeds were packed in an extractor fitted with bottom filter of suitable mesh size preferably 100 mesh so as not to allow the seed meal down along. With solvent Hexane is allowed to percolate through the packed fenugreek layer. The Percolated solvent is recycled efficiently over period of 8 to 10 hrs so that the resultant fenugreek meal is free of oils & lipids. The Hexane extracted meal is re-extracted with a solvent mixture comprising of aqueous aliphatic alcohol in the aqueous to alcohol ratio of 1:9 to 9:1 preferably 3:7 as the solvent. The said alcohol may be methyl alcohol, ethyl alcohol, isopropanol and preferably ethanol as the alcoholic solvent. The aqueous alcohol mixture is passed from top to bottom through the fenugreek layer in the percolater. The process of recycling the solvent was continued for a period of time ranging between 8 hrs to 10 hrs preferably 8 hrs at room temperature. The clear extract from the bottom of the percolater is inspected visually for any suspended particles and refiltered if necessary. The clear filtrate is vacuum concentrated at temperature ranging between 40° C. to 75° C. preferably at 55° C. to a pasty mass and the solvent recovered. The paste is redissolved in deionised water to a clear solution consisting of around 5% solid content. The clear solution is passed through an Ion Exchange resin column consisting of a strong acid anion in gel form to remove acidic compounds, amino acids, and other amphoteric compounds. The liquid from column outlet is collected in a stainless steel vessel.

This clear liquid is passed through an adsorbent resin column consisting of Highly porous adsorbent resin HP20SS or SP-207 beads column to bind the active compounds The column is monitored using a Thin layer chromatography system in which mobile Phase comprising of the solvents Toluene: ethylacetate: methanol: water in the ratio of 6:3:6:1 and the active principles are monitored by the spot development after spraying with methanolic sulphuric acid and heating the plate. The blakish brown glycoside spots were monitored for their absence in the outlet liquid.

After adsorption cycle the column is thoroughly washed with demineralised water and eluted with isopropanol in a gradient manner to elute all the compounds one after the other. The fractions in which the TLC fractions appealing at 0.6 Rf and 0.65 Rf are pooled together and concentrated under vacuum at 50° C. to get a semisolid paste. This paste is dissolved in 5 volume of water to get clear solution and spray dried to get free flowing powder. The process consists of the following steps:
Fenugreek seeds arc flaked to expose the inner core so as to ensure effective defatting, extraction and processing
Hexane solvent is passed through the fenugreek bed repeatedly to achieve effective defatting of the fenugreek.
The flaked seeds are loaded in a percolator and solvent mixture comprising of an aliphatic alcohol comprising of one carbon atom to three carbon atoms and water are passed through the fenugreek layer to achieve effective extraction of the, amino acids, saponin and other bioactive compounds.
The solvent is vacuum concentrated at lower temperatures to ensure the integrity of the mass and the resultant mass is dissolved in deionized water to get clear solution.
The clear solution thus obtained is passed through a strong acid cation exchange resin in Gel form to retain the amino acids and other acids.
The column outlet liquid is passed through an adsorbent column comprising of any one of the following commercial resins. SP-207, HP20SS, XAD-2 (halogenated).
The adsorbed material is eluted gradiently using aqueous alcohol to isolate the active compounds.

The above process is monitored by Thin layer chromatographic system comprising of toluene, ethylacetate, methanol, water in the ratio of 6:3:6:1.
The above isolated and purified material is spray dried into a free flowing powder.
The above purified material is subjected to HPLC measurement as per procedure defined hereunder to obtain 2 marker compounds. The main marker is to the extent of 70%.
The above extracted is further fractionated and purified to get a pure fraction of marker compound 1.
This market compound is subjected proton NMR and C-13 NMR to elucidate the structure of this glycoside.
The glycoside identified is 26-0-β-D Glucopyranosyl pseudodiosgenin-3-0-∝-L-Rhamnopyranosyl 1-(1-2)-β-D Glucopyranoside having a molecular weight of 914 and a chemical formula of C₄₇H₇₈O₁₇
This compound is tested for acute activity in Swiss Wistar rats to establish anti-inflammatory activity of 32.44% inhibition of paw edema in Carageneen induced models at 100 mg per kg dose.
This compound is tested in sub acute model of inflammation of cotton pallet granuloma in Swiss Wistar rats. This has an inhibition of granuloma of 32.3% at 100 mg per kg dosing.
This compound is tested in chronic model of arthritis of FCA (Freunds Complete Adjuvant) in Swiss Wistar rats. This has shown inhibition of 56% at 100 mg pet kg dose.
The compound is tested in chronic model of arthritis of FCA and has shown decrease in C-reactive protein indicative of reduction in inflammation.
The compound is tested for ulcer formation in Swiss Wistar rats and found that it does not produce any stomach ulcer.
This compound is tested in one year human clinical study in patients with rheumatoid arthritis. It has shown significant disease modifying anti-arthritic action accompanied by reduction in all ACR core set criteria, C-reactive protein and RA factor.
This compound has led to remission of disease in 10 patients treated during the clinical trial.
This compound has demonstrated a significant increase in HDL/LDL ratio of rheumatic patients from 0.41 to 0.45. Considering the fact that rheumatic patients develop cardiovascular problem, this drug is very beneficial in alleviating this.
The test compound has shown significant improvements in blood urea nitrogen and albumin confirming that the catabolic action of the rheumatoid arthritis is decreased

The technology of the instant Application is further elaborated with the help of following examples. However, the examples should not be construed to limit the scope of the invention.

### EXAMPLE NO: 1

500 gms of fenugreek seeds having a moisture content less than 5% were flaked in a roller flaker to a thickness of 2 mm. The flaked material is slacked in a column having a bed height of 200 mm. 2 liters of hexane was passed through the layer of fenugreek and the eluent collected from the bottom is recycled through the fenugreek layer for a period of 10 hrs at 35° C. After 10 hrs the fenugreek layer was drained free of hexane. Solvent mixture (4 liters) comprising of Ethyl alcohol and water in the ratio of 70:30 was passed through the layer for a period of 8 hrs at 35° C. by recycling the eluent. After 8 hrs the fenugreek bed is drained free of extracts and all the collected extracts were concentrated to semisolid mass under vacuum at 50° C. The concentrated mass is redissolved in 2 liters of deionized water to get a clear solution. The clear aqueous solution was passed through a column containing 200 ml of strong acid cation exchange Gel type resin for 2 hrs. TLC screening for the absence of Trigonelline and amino acids checked on the column eluent rising a system consisting of n-butanol: acetic acid: water in the ratio of 12:8:4 as the mobile phase on silica Gel precoated plate F254 (1.05554.007) and observation under UV 254 nm. The clear column outflow liquid devoid of all amino acids, proteins, Trigonelline, and other amphoteric compounds was passed again through a resin bed comprising of SEPABEADS SP-207 over a period of 2 hrs and the adsorption process monitored by thin layer chromatography system comprising of toluene; ethylacetate: methanol: water in the ratio of 6:3:0:1 resin beads washed thoroughly with demineralized water. The column was initially eluted with a mixture of water and ethyl alcohol in the ratio of 30:70 after about 2 bed volumes the ratio was changed to 10:90. The bioactive compounds as monitored by thin layer chromatographic system start eluting at this stage. These fractions were collected, screened and pooled together and concentrated at 50° C. to a paste. This paste was redissolved in water to get clear solution and spray dried under following condition.
Inlet temperature: 170° C.
Outlet temperature: 90° C.
Atomizer. RPM: 12000.

The yield 18 gms (The HPLC showed one major peak and two minor peaks apart from solvent methanol peak which was used for dissolving the compound before injection in to the system and the major peak area was around 70% area purity)

### EXAMPLE NO: 2

500 gms of fenugreek seeds having a moisture content less than 5% were flaked in a roller flaker to a thickness of 2 mm. The flaked material is slacked in a column having a bed height of 200 mm. 2 liters of hexane was passed through the layer of fenugreek and the eluent collected from the bottom is recycled through the fenugreek layer for a period of 10 hrs at 35° C. After 10 hrs the fenugreek layer was drained free of hexane. Solvent mixture (6 liters) comprising of Isopropyl alcohol and water in the ratio of 70:30 was passed through the layer for a period of 10 hrs at 30° C. by recycling the eluent. After 8 hrs the fenugreek bed is drained free of extracts and all the collected extracts were concentrated to semisolid mass under vacuum at 50° C. The concentrated mass is redissolved in 2 liters of deionised water to get a clear solution. The clear aqueous solution was passed through a column containing 200 ml of strong acid cation exchange Gel type resin for 2 hrs. TLC screening for the absence of Trigonelline and amino acids checked on the column eluent using a system consisting of n-Butanol: Acetic acid Water in the ratio of 12:8:4 as the mobile phase on silica Gel precoated plateF254(1.05554.007) and observation under UV 254 nm. The clear column outflow liquid devoid of all amino acids, proteins, Trigonelline, and other amphoteric compounds was passed again through a resin bed comprising of SEPABEADS SP-207 over a period of 2 hrs and the adsorption process monitored by thin layer chromatography system comprising of toluene: ethyl acetate: methanol: water in the ratio of 6:3: 6:1 resin beads washed thoroughly with demineralized water. The column was initially eluted with a mixture of water and isopropyl alcohol in the ratio of 30:70 after about 2 bed volumes the ratio was changed to 10:90. The bioactive compounds as monitored by thin layer chromatographic system starts eluting at this stage. These fractions were collected, screened and pooled together and concentrated at 50° C. to a paste. This paste was redissolved in water to get clear solution and spray dried under following condition:
Inlet temperature: 170° C.
Outlet temperature: 90° C.
Atomizer RPM: 12000.

The yield 15 gms (The HPLC showed one major peak, and two minor peaks apart from solvent. Methanol peak which was used for dissolving the compound before injection in to the system and the major peak area was around 70% area purity)

### EXAMPLE NO: 3

500 gms of fenugreek seeds having a moisture content less than 5% were flaked in a roller flaker to a thickness of 2 mm. The flaked material is stalked in a column having a bed height of 200 mm. 2 liters of hexane was passed through the layer of fenugreek and the eluent collected from the bottom is recycled through the fenugreek layer for a period of 10 hrs at 35° C. After 10 hrs the fenugreek layer was drained free of hexane. Solvent mixture (4 liters) comprising of Methyl alcohol and water in the ratio of 70:30 was passed through the layer for a period of 8 hrs at 35° C. by recycling the eluent. After 8 hrs the fenugreek bed is drained free of extracts and all the collected extracts the collected extracts were concentrated to semisolid mass under vacuum at 50° C. The concentrated mass is redissolved in 2 liters of deionized water to get a clear solution. The clear aqueous solution was passed through a column containing 200 ml of strong acid cation exchange Gel type resin for 2 hrs. TLC screening for the absence of Trigonelline and amino acids checked on the column eluent using a system consisting of n-Butanol: acetic acid: water in the ratio of 12:8:4 as the mobile phase on silica Gel precoated plate F254 (1.05554.007) and observation under UV 254 nm. The clear column outflow liquid devoid of all amino acids, proteins. Trigonelline, and other amphoteric compounds was passed again through a resin bed comprising of SEPABEADS SP-207 over a period of 2 hrs and the adsorption process monitored by thin layer chromatography system comprising of Toluene: Ethyl acetate: methanol: water in the ratio of 6:3: 6:1 resin beads washed thoroughly with demineralized water The column was initially eluted with a mixture of water and Methy alcohol in the ratio of 30:70 after about 2 bed volumes the ratio was changed to 10:90. The bioactive compounds as monitored by thin layer chromatographic system starts eluting at this stage. These fractions were collected, screened and pooled together and concentrated at 50° C. to a paste. This paste was redissolved in water to get clear solution and spray dried under following condition
Inlet temperature: 170° C.
Outlet temperature: 90° C.
Atomizer RPM: 12000.

The yield 14 gms (The HPLC showed one major peak and two minor peaks apart from solvent methanol peak which was used for dissolving the compound before injection in to the system and the major peak area was around 70% area purity).

### EXAMPLE NO: 4

500 gms of fenugreek seeds having a moisture content less than 5% were flaked in a roller flaker to a thickness of 2 mm. The flaked material is stalked in a column having a bed height of 200 mm. 2 liters of hexane was passed through the layer of fenugreek and the eluent collected from the bottom is recycled through the fenugreek layer for a period of 10 hrs at 35° C. After 10 hrs the fenugreek layer was drained free of hexane. Solvent mixture (4 liters) comprising of methyl alcohol and water in the ratio of 50:50 was passed through the layer for a period of 8 hrs at 35° C. by recycling the eluent. After 8 hrs the fenugreek bed is drained free of extracts and all the collected extracts the collected extracts were concentrated to semisolid mass under vacuum at 50° C. The concentrated mass is redissolved in 2 liters of deionized water to get a clear solution. The clear aqueous solution was passed through a column containing 200 ml of strong acid cation exchange Gel type resin for 2 hrs. TLC screening for the absence of Trigonelline and amino acids checked on the column eluent rising a system consisting of n-Butanol: Acetic acid: Water in the ratio of 12:8:4 as the mobile phase on silica Gel precoated plateF254(1.05554.007) and observation under UV 254 nm. The clear column outflow liquid devoid of all amino acids, proteins, Trigonelline, and other amphoteric compounds was passed again through a resin bed comprising of SEPABEADS SP-207 over a period of 2 hrs and the adsorption process monitored by thin layer chromatography system comprising of toluene: ethyl acetate: methanol: water in the ratio of 6:3: 6:1 resin beads washed thoroughly with demineralized water. The column was initially eluted with a mixture of water and Methyl alcohol in the ratio of 50:50 after about 2 bed volumes the ratio was changed to 20:80. The bioactive compounds as monitored by thin layer chromatographic system starts eluting at this stage. These fractions were collected, screened and pooled together and concentrated at 50° C. to a paste. This paste was redissolved in water to get clear solution and spray dried under following condition.
Inlet temperature: 170° C.
Outlet temperature: 90° C.
Atomizer RPM: 12000

The yield 15 gms (The HPLC showed one major peak and two minor peaks apart from solvent methanol peak which was used for dissolving the compound before injection in to the system and the major peak area was around 70% area purity).

### EXAMPLE NO: 5

500 gms of fenugreek seeds having a moisture content less than 5% were flaked in a roller flaker to a thickness of 2 mm. The flaked material is stalked in a column having a bed height of 200 mm. 2 liters of hexane was passed through the layer of fenugreek and the eluent collected from the bottom is recycled through the fenugreek layer for a period of 10 hrs at 35° C. After 10 hrs the fenugreek layer was drained free of hexane. Solvent mixture (4 liters) comprising of Ethyl alcohol and water in the ratio of 50:50 was passed through the layer for a period of 8 hrs at 35° C. by recycling the eluent. After 8 hrs the fenugreek bed is drained free of extracts and all the collected extracts the collected extracts were concentrated to semisolid mass under vacuum at 50° C. The concentrated mass is redissolved in 2 liters of deionized water to get a clear solution. The clear aqueous solution was passed through a column containing 200 ml of strong acid cation exchange Gel type resin for 2 hrs. TLC screening for the absence of Trigonelline and amino acids checked on the column eluent using a system consisting of n-Butanol: Acetic acid Water in the ratio of 12:8:4 as the mobile phase on silica Gel precoated plateF254 (1.05554.007) and observation under UV 254 nm. The clear column outflow liquid devoid of all amino acids, proteins, Trigonelline, and other amphoteric compounds was passed again through a resin bed comprising of HP20SS Beads over a period of 2 hrs and the adsorption process monitored by thin layer chromatography system comprising of Toluene; Ethyl acetate: methanol: water in the ratio of 6:3: 6:1 resin beads washed thoroughly with demineralised water. The column was initially eluted with a mixture of water and Ethyl alcohol in the ratio of 50:50 after about 2 bed volumes the ratio was changed to 20:80. The bioactive compounds as monitored by thin layer chromatographic system starts eluting at this stage. These fractions were collected, screened, and pooled together and concentrated at 50° C. to a paste. This paste was redissolved in water to get clear solution and spray dried under following condition:
Inlet temperature: 170° C.
Outlet temperature: 90° C.
Atomizer RPM: 12000.

The yield 16 gms. (The HPLC showed one major peak and two minor peaks apart from solvent methanol peak which was used for dissolving the compound before injection in to the system and the major peak area was around 70% area purity)

### EXAMPLE NO: 6

500 gms of fenugreek seeds having a moisture content less than 5% were flaked in a roller flaker to a thickness of 2 mm. The flaked material is stalked in a column having a bed height of 200 mm. 2 liters of hexane was passed through the layer of fenugreek and the eluent collected from the bottom is recycled through the fenugreek layer for a period of 10 hrs at 35° C. After 10 hrs the fenugreek layer was drained free of hexane. Solvent mixture (4 liters) comprising of Isopropyl alcohol and water in the ratio of 50:50 was passed through the layer for a period of 8 hrs at 35° C. by recycling the eluent. After 5 hrs the fenugreek bed is drained free of extracts and all the collected extracts the collected extracts were concentrated to semisolid mass under vacuum at 50° C. The concentrated mass is redissolved in 2 liters of deionized water to get a clear solution. The clear aqueous solution was passed through a column containing 200 ml of strong acid cation exchange Gel type resin for 2 hrs. TLC screening for the absence of Trigonelline and amino acids checked on the column eluent using a system consisting of n-Butanol: Acetic acid. Water in the ratio of 12:8:4 as the mobile phase on silica Gel precoated plateF254 (1.05554.007) and observation under UV 254 nm. The clear column outflow liquid devoid of all amino acids, proteins, Trigonelline, and other amphoteric compounds was passed again through a resin bed comprising of XAD-2 brominated beads over a period of 2 hrs and the adsorption process monitored by thin layer chromatography system comprising of Toluene: Ethyl acetate: methanol: water in the ratio of 6:3: 6:1 resin beads washed thoroughly with demineralised water. The column was initially eluted with a mixture of water and Isopropyl alcohol in the ratio of 50:50 after about 2 bed volumes the ratio was changed to 20:80. The bioactive compounds as monitored by thin layer chromatographic system starts eluting at this stage. These fractions were collected, screened and pooled together and concentrated at 50° C. to a paste. This paste was redissolved in water to get clear solution and spray dried under following condition.
Inlet temperature: 170° C.
Outlet temperature: 90° C.
Atomizer RPM: 12000.

The yield 16 gms (The HPLC showed one major peak and two minor peaks apart from solvent methanol peak which was used for dissolving the compound before injection in to the system and the major peak area was around 70% area purity).

### EXAMPLE NO: 7

HPLC of the purified extract is performed as per the system specified below.

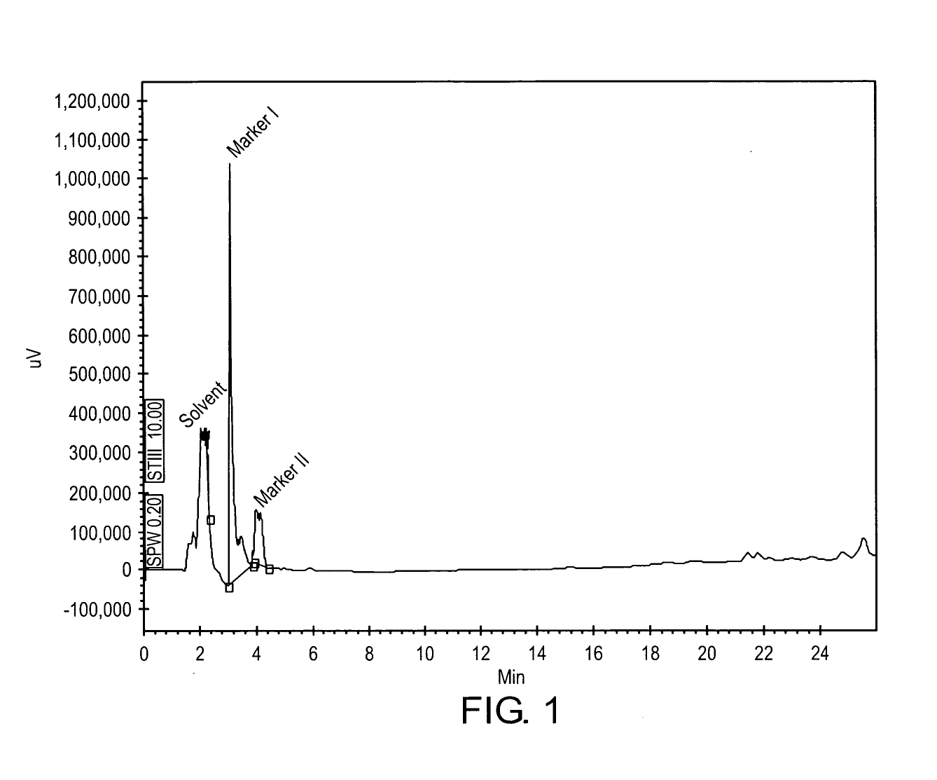

Two maker compounds are obtained. The marker compound which elutes at the retention time (RT) of 3 minutes is named as marker 1 and this accounts for about 70% of the assay. This is a glycoside. The marker 2 elutes at RT of 4 minutes and accounts for about 20%. This is also a closely resembling glycoside. The HPLC chromatogram is shown in FIG. 1. The HPLC conditions include column 250 mm×4.6 mm, 5 micron, e-18, Mobile phase: water: acetonitrile (75:25) gradient technique 65:35 in 20 minutes at a flow rate of 1.0 ml/min at a wavelength of 205 nm.

TABLE 1
HPLC chromatographic data for the two marker compounds

| | | Start | Time | End | Ret. time Offset | Quantity | Height | Area | Area % |
|---|---|---|---|---|---|---|---|---|---|
| Index | Name | [Min] | [Min] | [Min] | [Min] | [% Area] | [uV] | [uV · Min] | [%] |
| 3 | Solvent | 2.199 | 2.275 | 2.362 | 0.000 | 4.78 | 114847.6 | 9172.7 | 4.781 |
| 1 | Marker I | 3.010 | 3.100 | 3.865 | 0.000 | 73.48 | 1163467.3 | 140985.1 | 73.478 |
| 2 | Marker II | 3.888 | 4.017 | 4.433 | 0.000 | 21.74 | 146931.6 | 41715.3 | 21.741 |
| Total | | | | | | 100.00 | 1425246.5 | 191873.0 | 100.00 |

### EXAMPLE NO: 8

The extract was further fractionated on a sephadex column to separate the marker 1 and marker 2 fractions. Fraction 1 thus collected and screened for the major saponin was further crystallized in methanol to get a crystalline material the 13-C NMR DATA (in pyridine D-6) is as follows corresponding to the Furastanol glycoside of the following structure

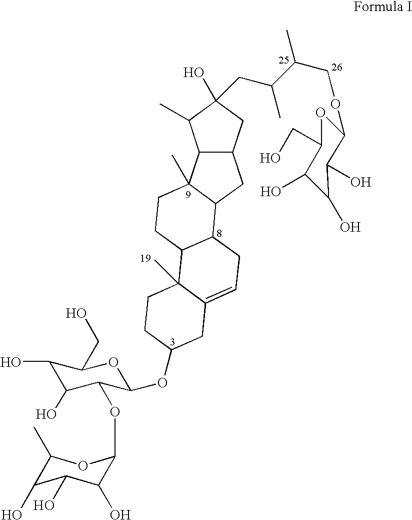

The saponin is identified as 26-0-β-D Glucopyranosyl pseudodiosgenin-3-0-∝-L-Rhamnopyranosyl 1-(1-2)-β-D Glucopyranoside.

Molecular Formula: C₄₇H₇₈O₁₇

Molecular weight: 914

TABLE NO: 2
NMR data for aglycon (saponin) moiety

| NO | PPM |
|---|---|
| 1 | 37.5 |
| 2 | 30 |
| 3 | 78 |
| 4 | 39 |
| 5 | 140 |
| 6 | 121 |
| 7 | 32 |
| 8 | 31 |
| 9 | 50 |
| 10 | 37 |
| 11 | 21 |
| 12 | 40 |
| 13 | 40.5 |
| 14 | 56.5 |
| 15 | 31.5 |
| 16 | 81 |
| 17 | 64 |
| 18 | 16 |
| 19 | 19 |
| 20 | 40.5 |
| 21 | 16.5 |
| 22 | 110 |
| 23 | 31 |
| 24 | 28 |
| 25 | 34 |
| 26 | 75 |
| 27 | 17.5 |

### EXAMPLE NO. 9

### Acute Study

These tests are for the inhibition of inflammation caused by prostaglandins in short period of time. This can be tested in animal (Swiss Wistar rats) as per well known protocols which are treated with Carrageenan. This model tests out anti-inflammatory activity caused by prostaglandin inhibition. Celecoxib is given as a positive control to do a comparison and evaluate the efficacy of anti-inflammatory purified extract as described above in Prostaglandin inhibition in 3 hrs.

TABLE NO: 3
Anti-inflammatory activity for test compound and its comparison with
fenugreek seed powder and alcoholic extract is shown

| | | Anti-inflammatory activity |
|---|---|---|
| | | (% Inhibition) in |
| | | Carrageenan model |
| Treatment | Dose mg/kg | In 3 Hrs. |
| Control | — | — |
| Fenugreek seed powder | 2000 | 8.2 |
| Fenugreek alcohol extract | 500 | 18.1 |
| Test compound | 50 | 26.49* |
| | 100 | 32.54* |
| | 200 | 34.44* |
| Celecoxib | 10 | 45.2** |

One way ANOVA followed Dunnett's test,
*P < 0.05,
**P < 0.01,
N = 6

Paw Volume measured is compared with Control group and standard drug, celecoxib. Paw volume initial to the paw volume at 1 h, 2 h, 3 h is measured and peak inhibition, which is at 3 hrs is noted down. In this model efficacy as well as dosage of drug is arrived by considering peak % inhibition. Fenugreekseed powder at a high dose of 2000 gm per kg body weight induced 8.2% inhibition. This is not statistically significant for any action, fenugreek seed alcohol extract dry powder created an inhibition of 18.1%) at a high dose of 500 mg per kg body weight. This is also not statistically significant. The test compound performed well at doses of 100 mg per kg and 200 mg per kg with statistical significance and is comparable with Celecoxib action 100 mg per kg is the optimal dose as 200 mg per kg is not showing remarkable increase in activity.

### EXAMPLE NO. 10

### Sub Acute Study

This evaluates inflammation over a period of 7 days. This is done by classical animal model (in Swiss Wistar tats) of cotton pallet granuloma. Celecoxib is a positive control for comparison of the efficacy of Test Compound.

TABLE NO: 4
Anti-inflammatory activity for test compound and its comparison with
fenugreek seed powder and alcoholic extract performed by cotton
pellet granuloma

| | | Anti-inflammatory activity |
|---|---|---|
| | | (% Inhibition) in |
| Treatment | Dose mg/kg | Cotton pellet Granuloma |
| Control | — | — |
| Fenugreek seed powder | 2000 | 6.1 |
| Fenugreek alcohol extract | 500 | 14.6 |
| Test compound | 50 | 24.2* |
| | 100 | 33.2* |
| | 200 | 39.4** |
| Celecoxib | 10 | 43.2** |

One way ANOVA followed Dunnett's test,
*P < 0.05,
**P < 0.01,
N = 6

Implantation of cotton pellets produced granuloma formation, which was measured by weighing the dried pellets after 7 days of implantation and treatment Fenugreek seed powder at a high dose of 2000 mg per kg of body weight produced statistically insignificant inhibition of 6.1%. Fenugreek alcohol extract also at a high dose of 500 mg per kg of body weight did not have significant activity. Dose dependent percent inhibition of granuloma formation was observed in Test compound (50, 100 and 200 mg/kg) and Celecoxib (10 mg/kg) treated groups. The test compound showed statistically significant result at 100 mg per kg and 200 mg per kg doses. 100 mg per kg is the optimal dose. A comparison is made with Celecoxib.

### EXAMPLE NO. 11

### Chronic Study

This activity mimics the onset of autoimmune damage to joints and tests the efficiency of drug against this damage. Leflunamide is taken as positive control. Here Swiss Wistar rats are treated with Freunds Complete Adjuvant (CFA) to induce chronic inflammation symptoms similar to rheumatis diseases. The symptoms appear by 13^{th} day. The drug treatment is started from 13^{th} day to 23^{rd} day for 10 days.

TABLE 5
Anti-inflammatory activity of test compound by FCA induced
arthritis and its comparison with fenugreek seed powder and
alcoholic extract

| | | Anti-inflammatory | |
|---|---|---|---|
| | | activity | |
| | | (% Inhibition) | |
| | | in FCA induced | |
| | | arthritis | |
| Treatment | Dose mg/kg | Day 18 | Day 23 |
| Control | — | — | — |
| Fenugreek seed powder | 2000 | 4.3 | 6.2 |
| Fenugreek alcohol extract | 500 | 16.8 | 20.6 |
| Test compound | 50 | 18.04 | 29.07* |
| | 100 | 32.38 | 52.81* |
| | 200 | 25.41 | 56.55* |
| Leflunamide | 10 | 35.22* | 76.55** |

One way ANOVA followed Dunnett's test,
*P < 0.05,
**P < 0.01,
N = 6

Fenugreek seed powder at a high dose of 2000 mg per kg body weight did not have any significant activity, nor did the alcohol extract at a high dose of 500 mg per kg body weight.

A few hours after the inoculation of adjuvant arthritis by sub planter injection of Freunds Complete Adjuvant (FCA) to rats, the animals showed a local inflammatory reaction in the injected paw (primary response) with an increase in planter volume of about the 60% over the baseline value. In addition, a disseminated arthritic reaction (secondary response) developed from day 1 alter FCA injection. The swelling was observed both in the injected paw and in the non-injected contra lateral paw. The reduction of the planter volume was statistically significant for both hind legs. The test compound gave a very impressive reduction of 52% at 100 mg per kg dose which is statistically significant. This compared very well with DMARD drug Leflunomide. However, Leflunomide is highly nephrotoxic and cannot be administered for a long time. This is here the Test compound has a good advantage as this drug does not have any toxicity in long term usage.

### EXAMPLE NO: 12

### C—Reactive Protein

TABLE 6
C-reactive protein results for test compound

C-reactive protein

| | | After 10 Day |
|---|---|---|
| Group | Before Treatment | Treatment |
| Vehicle | Negative | Positive |
| Fenugreek seed powder | Negative | Positive |
| Fenugreek alcohol extract | Negative | Positive |
| Test Compound | Negative | Negative |
| Leflunomide | Negative | Negative |

It is very important to note that the Fenugreek seed powder and Alcohol extract did not eliminate Creactive protein. In this method the CRP test kit (Plasmatec, Dreadnought Trading Estate Bridgeport Dorset DT6 5BU UK) was used for the measurement of CRP. The results are expressed either -1 ve or -ve depending on the formation of precipitate Normal range of CRP: up to 6 mg/L. Below this level is considered to be negative.

### EXAMPLE NO: 13

Effect of Test compound and standard drug Celecoxib on TNF-α level in FCA induced arthritis rat. The serum from the FCA induced arthritic rat was examined for Tumor necrosis factor alpha. The experimental protocol was same as example no. 11:

TABLE 7
TNF data for test compound

| | Concentration of | | |
|---|---|---|---|
| | TNF-α in pg/mL | | |
| Group | Day 0 | Day 13 | Day 21 |
| Control | 20.1 | 49.1 | 52.3 |
| Celecoxib | 28.9 | 47.9 | 33.6 |
| Test compound | 36.8 | 45.3 | 36.1 |

This clearly shows that the experimental drug inhibits TNF-α on day 21. TNF-α is involved in synovial membrane damage. The test drug has a disease modifying activity.

### EXPERIMENT NO. 14

### Gastric Ulcerogenic Effects of Test Compound

Gastric ulcerogenic effects of Test compound and celecoxib after repeated oral administration for 7 consecutive days to the male Swiss Wistar rats.

TABLE 8
Gastric ulcerogenic effect test results of test compound

| | Dose | Average of | Mean score | No. of | Ulcer |
|---|---|---|---|---|---|
| Group | (mg/kg, | no. of ulcer | of Lesion | animals with | Index |
| Treatment | p.o.) | per animal | intensity | ulcer (%) | (UI) |
| Control | Nil | Nil | Nil | Nil | Nil |
| Celecoxib | 5 | 2 | 1.6 | 2 (33.3) | 3.7 |
| Test drug | 200 | Nil | Nil | Nil | Nil |

In the control group, structure of the gastric mucosa appears normal. In the group treated with Test drug, the surface epithelium was also continuous and normal. In the group treated with Celecoxib showed mild gastric ulcers in all the animals under treatment. The test drug does not have any gastric ulcerogenicity.

### EXAMPLE NO. 15

### Human Clinical Study

### An Overview

This is one year open label human clinical study conducted for 1 year
70 subjects conforming to inclusions criteria recruited for the study and 58 completed the study at the end of 12 months.
This study was conducted as per ICH/GCP guideline with informed consents as per Hilsinki Protocol.
The test compound was given at the rate of 16 mg per kg of body weight. This was equivalent to 500 mg capsules twice daily halt an hour before meals.
NO NSAIDs, DMARDs, or STEROIDs were permitted during the entire one year study duration.
Paracetamol was the only analgesic allowed to be used on a sos basis and its consumption was monitored
Medication for concomitant illness was permitted during the study
No dietary or any kind of life style modification were encouraged
ACR core set of efficacy measures as
   JCPT (Pain VAS), Joint count for Pain and tenderness.
   JCSW Joint count for swelling
   Pain VAS
   Patient and physician global assessment.
   HAQ
   and ESR

Were measured at baseline, Week 2, Week 4, Week 8, and monthly thereafter till Week 52 as per the protocol using a standard trial CRT
ACR 20 & 50 therapeutic responses were computed
Patients were questioned at: every visit for predetermined likely side effects check list
Adverse Events (AE) were recorded as per GCP guidelines in the CRP
The sample size was designed with 80% power (to detect 20% difference between active and placebo) and 5% Type 1 error (significance at p<0.05) and a presumed drop out rate of 20%, as per accepted norms.
Intent to—treat analysis, with last observation carried forward, at a significance of p≦0.05 was done.

### Inclusion Criteria:

Patient of either sex 18 years and above
Rheumatoid Arthritis as defined by 4 of the 7 revised criteria of American College of Rheumatology classification of RA
Acute inflammatory state defined by at least 2 of the following: P2 Morning stiffness for 60 mins
   ESR≧30 mm
   3 inflamed joints
   6 painful joints
Patients classified as ACR functional class I and II
No herbal treatment for past 1 month
No ongoing treatment with DMARDS or glucocorticoids for 4 months preceding the trial
No history of allergy to the ingredients of test compound plant derivatives
Informed consent and willing for follow-up

### Efficacy Measures:

I—PRIMARY
Joint count (as per ACR) for pain/tenderness (JCPT) for 68 joints
Joint count (as per ACR) for swelling (JCSW) for 66 joints
Pain V AS: A horizontal numerical rating scale 0-10 cms, marked at every 1 cm.
Patient global assessment: A horizontal numerical rating scale, 0-10 cms, marked at every 1 cm.
Physician global assessment: A horizontal numerical rating scale, 0-10 cms, marked at every 1 cm
HAQ (Health Assessment Questionnaire)
ESR (WESTERGREN)

### II—SECONDARY

Morning stiffness (or following a period of inactivity)
Walking time: The time taken by the patient to walk (as rapidly as possible) on a straight continuous horizontal distance of 50 feet
Grip Strength: With the mercury bulb inflated up to 20 mm, the patient was asked top grip it as tightly as possible with both hands separately.
Rheumatoid factor and C Reactive protein measured by Nepblometry assay.

### SAFETY

a. Complete blood count, Liver function tests, Lipid profile. Kidney function test and urine analysis was carried out at baseline, 12 weeks, 24 weeks, 36 weeks and 52 weeks.
b. ESR was carried out at each endpoint
c. Women in reproductive age group were tested for Urine pregnancy test at baseline, 12 weeks, 24 weeks, 36 weeks and 52 weeks.

### Abbreviation:

JCPT: Joint Count Pain/Tenders
JCSW: Joint count swelling
phy: physician
pat: patient
Global: Global disease activity
painVAS: pain visual analogue scale
HAQ: Health Assessment Questionnaire
mths: month
NS: not statistically significant
act: active
RF: rheumatoid factor titer (IU/ml)
CRP: C-Reactive Protein
N: Number of patients

### One Year Study Results

70 patients entered the one year human study and 58 patients completed one year.
Significant improvement (P<0.05) was seen in all the clinical efficacy measures
ACR 20 & ACR 50 improvement response seen in 80% and 40% patients respectively.
10 (11.9%) patients out of the total 70 patients recruited in CRD became completely asymptomatic as per the ACR core set clinical measures (including HAQ) and remained so for varying time periods till completion of the study Table2.
5 patients satisfied the ACR criteria for remission that also includes the ESR response

### Primary Efficacy Parameters

TABLE 9
One year study: Mean change in efficacy measures

| | Baseline | 12 mths | | 95% CI of |
|---|---|---|---|---|
| Variable | (mean) | (mean) | Mean change | Mean change |
| JCPT | 24.3 | 5.5 | 18.8 | 15.7, 21.8* |
| JCSW | 9.5 | 1.6 | 7.9 | 6.2, 9.5* |
| painVAS | 6.6 | 3.6 | 3.0 | 2.5, 3.5* |
| patGlobal | 6.4 | 3.4 | 3.0 | 2.5, 3.5* |
| phyGlobal | 5.7 | 2.6 | 3.10 | 2.6, 3.5* |
| HAQ | 8.8 | 4.4 | 4.35 | 3.3, 5.4* |
| ESR | 61.4 | 66 | −3.05 | −10.6, 4.5 |

*P < 0.05 (Mann Whitney)

TABLE 10
Patients in Clinical Remission

| | | Duration |
|---|---|---|
| Sr. No. | Patient No. | (Months) |
| 1 | 019 | 8 |
| 2 | 035* | 3 |
| 3 | 037* | 3 |
| 4 | 038* | 3 |
| 5 | 049 | 5 |
| 6 | 051 | 2 |
| 7 | 054* | 3 |
| 8 | 057 | 6 |
| 9 | 059 | 5 |
| 10 | 112* | 7 |

*ACR REMISSION; ESR <30 mm

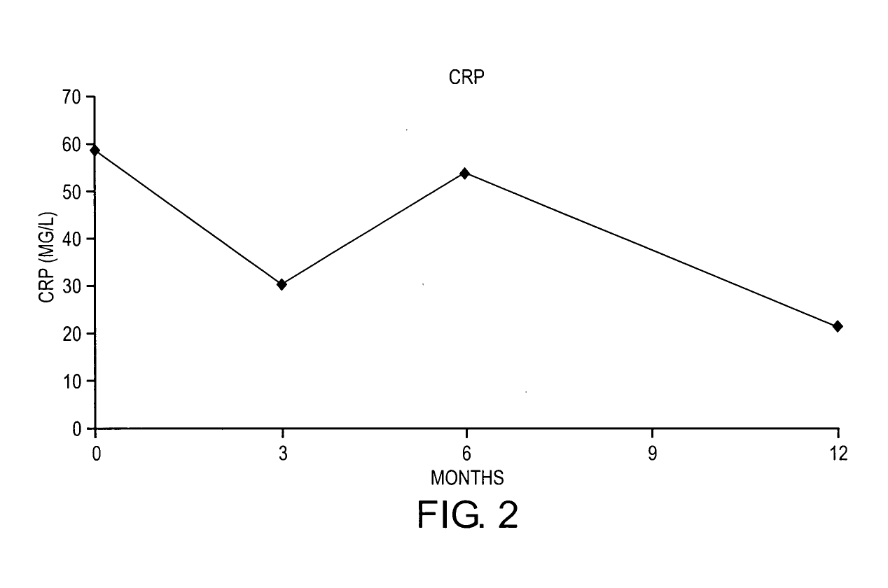

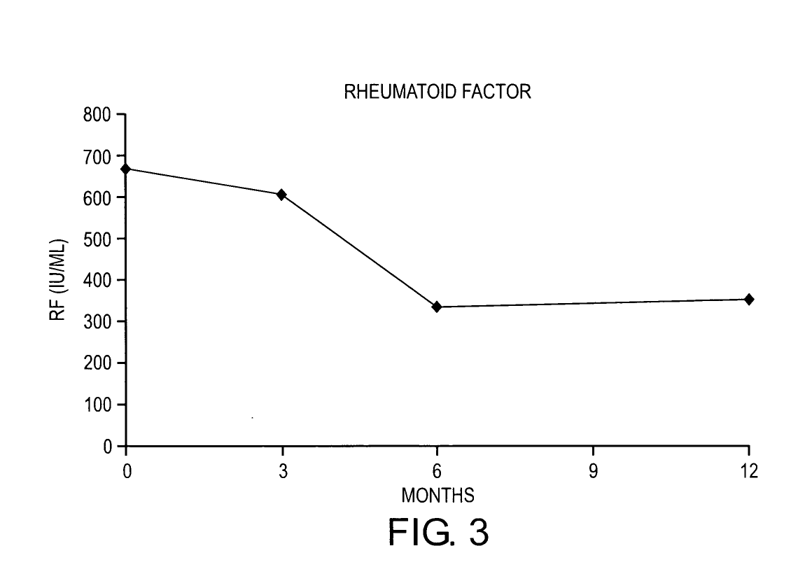

FIG. 2 and 3 show the mean nephlometric values of CRP (C Reactive Protein) and RF (Rheumatoid factor) Titre over time; the reduction by 12 months is significant.

### Safety:

Minor side-effects, often observed in Indian clinical practice and requiring short term symptomatic medication, were recorded during the entire study period
There were no significant adverse alterations in the routine haematology, biochemistry (especially renal and hepatic) and metabolic parameters attributable to test compound in 12 months study.

### Other Interesting Results

A significant increase in the HDL/LDL ratio was observed in study group.
No significant changes were seen in the laboratory values for SGOT, SGTP, GGT, Serum bilirubin and Serum creatinine at 12 months from baseline.
Increase in the serum protein with significant increase in serum albumin (95% CI—0.35, −2.90) was observed at 12 months.
Significant decrease in Blood Urea Nitrogen (95%) CI 1.2, 35) was observed at 12 months.
BUN and Albumin show increase in anabolic activity and reduction in disease activity which breaks down joint tissue.

TABLE 11
Mean Change in Lipids

| | BASELINE | | MEAN CHANGE | | |
|---|---|---|---|---|---|
| | Test | | Test | | DIFFERENCE |
| | Compound | Placebo | Compound | Placebo | 95% CI |
| HDL | 44.6 | 45.58 | −1.57 | 1.7 | −0.41, 7.12 |
| LDL | 117.08 | 107.83 | 12.7 | 4.19 | −22.0, 4.94 |
| HDL/ | 0.41 | 0.47 | −6.1 | 2.88 | 0.01, 0.17* |
| LDL | | | | | |

*P < 0.05

### Conclusion

1. Test compound showed impressive improvement in all joint related measurements as per ACR.
2. Test compound has showed 80% responders for ACR 20 criteria improvement. That is 20% improvement in ACR factors.
3. Test compound has showed 40% responders for ACR 50 criteria improvement.
4. Test compound has shown significant improvement in joint count pain and tender, joint count swelling, and pain visual analog scale.
5. Test compound has shown significant improvement, in patient: global assessment.
6. Test compound has shown significant improvement in physician global assessment.
7. Test compound has shown significant improvement in health assessment questionnaire.
8. Test compound has shown significant improvement in C-reactive protein and Rheumatoid factor
9. Test compound has shown significant improvement in HDL/LDL ratio.
10. Test compound has shown significant improvement in BUN and Albumin indicating reduction in tissue breakdown activities.

We claim:

1. A compound of structural formula I

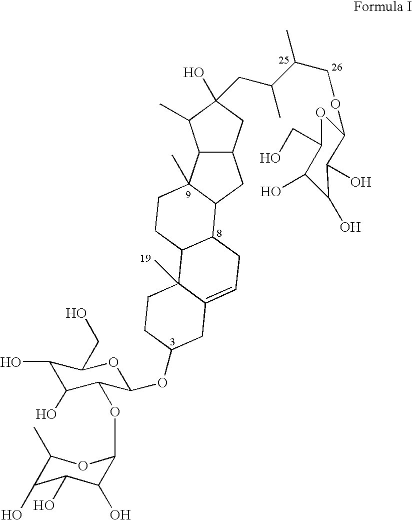

2. The compound as claimed in claim 1, wherein said compound is chemically 26-0-β-D Glucopyranosyl pseudodiosgenin-3-0-α-L- Rhamnopyranosyl 1-(1-2)-β-D Glucopyranoside.

3. The compound as claimed in claim 1, wherein said compound is a steroidal glycoside having molecular weight of 914 and molecular formula of C₄₇H₇₈O₁₇

4. The compound as claimed in claim 1, wherein said compound is obtained from plant Trigonella foenum graecum.

5. A composition comprising compound of structural formula I 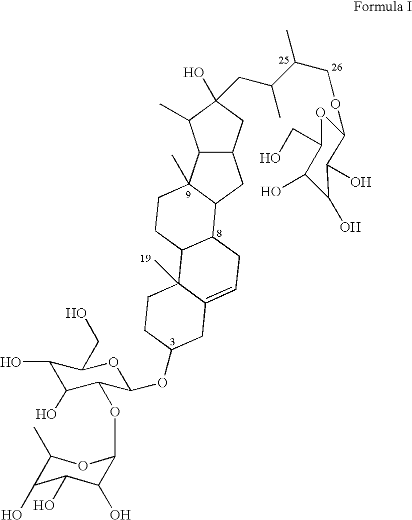
and pharmaceutically acceptable additive(s) for management of rheumatic and inflammatory disease conditions.

6. The composition as claimed in claim 5, wherein the additives are selected from a group comprising granulating agents, binding agents, lubricating agents, disintegrating agents, sweetening agents, coloring agents, flavoring agents, coating agents, plasticizers, preservatives, suspending agents, emulsifying agents and spheronization agents.

7. The composition as claimed in claim 5, wherein said composition is formulated into various dosage forms selected from a group comprising tablet, troches, lozenges, aqueous or oily suspensions, dispersible powders or granules, emulsion in hard or soft gel capsules, syrups, elixirs, phyotceuticals, neutraceuticals and food stuffs.

8. The composition as claimed in claim 5, wherein said compound is obtained from plant Trigonella foenum graecum.

9. The composition as claimed in claim 8, wherein said plant parts are roots, shoots, leaves, seeds, entire plant, and preferably seeds.

10. The composition as claimed in claim 5, wherein said composition is non-toxic and free of side effects.

11. A process for preparation of compound of structural formula I, 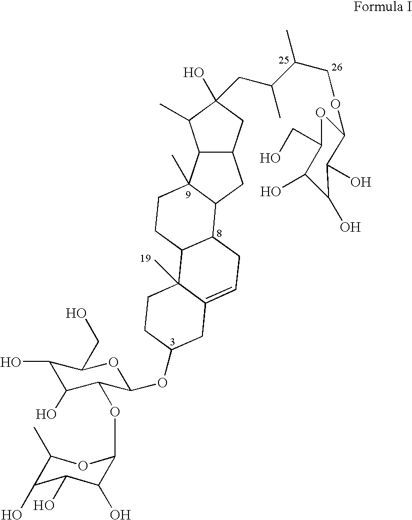
from plant Trigonella foenum-graecum, wherein said process comprises steps of
a. flaking fenugreek seeds;
b. extracting flaked seeds with n-hexane and hydro-alcohol to remove fatty matter;
c. concentrating the extract under vacuum to obtain a mass;
d. dissolving concentrated mass to obtain a clear solution;
e. passing clear solution through ion-exchange resin and adsorbent column to retain active compounds; and
f. eluting adsorbed active compounds using hydro-alcohol and drying the eluant to obtain powder of the compound of formula I.

12. The process as claimed in claim 11, wherein said extraction is carried out for a time period ranging from 8 to 12 hrs and preferably 10 hrs.

13. The process as claimed in claim 11, wherein said extraction is carried out at a temperature ranging from 30 to 40° C. and preferably 35° C.

14. The process as claimed in claim 11, wherein the hydro alcohol for extraction is isopropyl alcohol and water in a ratio ranging from 50:50 to 80:20 and preferably 70:30.

15. The process as claimed in claim 11, wherein said extract is concentrated under vacuum at a temperature ranging from 45 to 55° C. and preferably 50° C.

16. The process as claimed in claim 11, wherein said mass is dissolved in deionized water.

17. The process as claimed in claim 11, wherein said ion-exchange resin is cation exchange gel type resin.

18. The process as claimed in claim 11, wherein said hydro alcohol for eluting adsorbed compounds is water and ethyl alcohol at an initial ratio of 30:70 followed by a shift to ratio of 10:90.

19. The process as claimed in claim 11, wherein the yield of the compound of formula 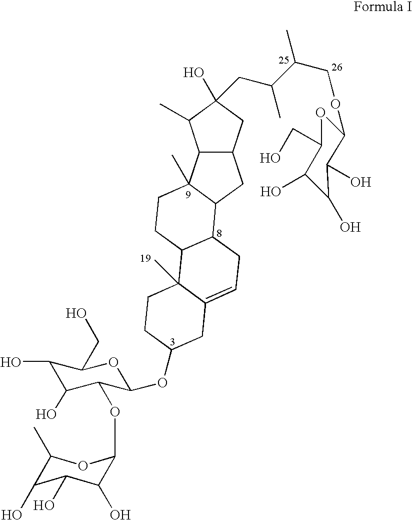
is ranging between 12 to 20 gms/500 gm of fenugreek seeds.

20. The process as claimed in claim 11, wherein the purity of the compound of formula I is ranging from 50% to 70%.

21. A method of treating rheumatic and inflammatory disease conditions in a subject in need thereof, said method comprising step of administering pharmaceutically effective amount of compound of structural formula I 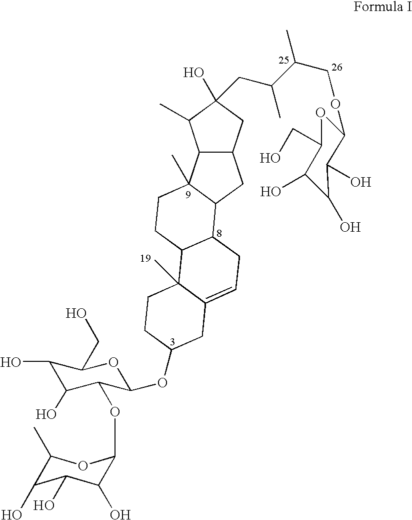
optionally along with pharmaceutically acceptable additives to the subject.

22. The method of treating as claimed in claim 21, wherein said compound is effective in reducing disease related factors comprising Rheumatoid factor, Tumor Necrosis Factor-α, Blood Urea Nitrogen, and C-reactive protein and increasing Albumin, HDL/LDL ration.

23. The method of treating as claimed in claim 21, wherein said additives are selected from a group comprising granulating agents, binding agents, lubricating agents, disintegrating agents, sweetening agents, coloring agents, flavoring agents, coating agents, plasticizers, preservatives, suspending agents, emulsifying agents and spheronization agents.

24. The method of treating as claimed in claim 21, wherein said composition is formulated into various dosage forms selected from a group comprising tablet, troches, lozenges, aqueous or oily suspensions, dispersible powders or granules, emulsion in hard or soft gel capsules, syrups, elixirs, phyotceuticals, neutraceuticals and food stuffs.

25. The method of treating as claimed in claim 21, wherein the subject is an animal or human being.

26. The method of treating as claimed in claim 21, wherein the composition is administered at dosage ranging between 5 mg/kg to 20 mg/kg body weight and preferably 16 mg/kg body weight.

27. The method of treating as claimed in claim 21, wherein the composition is non-toxic and free of adverse effects including gastric ulcerogenicity.

28. The method of treating as claimed in claim 21, wherein said compound is obtained from plant Trigonella foenum graecum.

29. The method of treating as claimed in claim 28, wherein said compound is obtained from plant parts are tools, shoots, leaves, seeds, entire plant and preferably seeds.

30. The method of treating as claimed in 21, wherein said rheumatic disease conditions comprise bursitis, tendonitis, rheumatoid arthritis, osteoarthritis, soft tissue rheumatism, spondylitis and back pain.
